(19) United States
(12) Patent Application Publication
(10) Pub. No.: US 2017/0209062 A1
(43) Pub. Date: Jul. 27, 2017

(54) MAGNETIC SENSOR AND MAGNETIC SENSOR APPARATUS

(71) Applicant: KABUSHIKI KAISHA TOSHIBA, Tokyo (JP)

(72) Inventors: Hitoshi IWASAKI, Tokyo (JP); Akira KIKITSU, Yokohama (JP); Satoshi SHIROTORI, Yokohama (JP)

(21) Appl. No.: 15/266,352

(22) Filed: Sep. 15, 2016

(30) Foreign Application Priority Data
Jan. 26, 2016 (JP) 2016-012662

(51) Int. Cl. A61B 5/04; H01L 43/10; H01L 43/08

(52) U.S. Cl. CPC A61B 5/04007; H01L 43/08; H01L 43/10; A61B 5/04008; A61B 2562/0223

(57) ABSTRACT

A magnetic sensor according to an embodiment includes: a magneto-resistive film including a laminate structure, the laminate structure including a first magnetic layer, a second magnetic layer, and an intermediate layer arranged between the first magnetic layer and the second magnetic layer; and a pair of electrodes for supplying current in a first direction perpendicular to a laminate direction of the magneto-resistive film, wherein the second magnetic layer includes an amorphous magnetic layer, and a crystalline magnetic layer arranged between the amorphous magnetic layer and the intermediate layer, and a length of a current path of the magneto-resistive film is 10 μm or more.

### CROSS REFERENCE TO RELATED APPLICATIONS

This application is based upon and claims the benefit of priority from prior Japanese Patent Application No. 2016-012662 filed on Jan. 26, 2016 in Japan, the entire contents of which are incorporated herein by reference.

### FIELD

Embodiments described herein relate generally to a magnetic sensor and a magnetic sensor apparatus.

### BACKGROUND

Conventionally, a biomagnetic measurement apparatus using a magnetic sensor having a superconducting quantum interference device (SQUID) have been devised as an apparatus for measuring a magnetic field generated from a living body. The biomagnetic measurement apparatus is capable of obtaining two-dimensional biomagnetic information such as a magnetoencephalogram, or a magnetocardiogram, by arraying multiple SQUID magnetic sensors and using the sensors for biomagnetic measurement. Since the SQUID magnetic sensor uses superconductivity and is required to be kept in an extremely low temperature state, it is necessary to cool with a refrigerant such as liquid helium. For this reason, the SQUID magnetic sensor has a problem that its cost is increased.

To solve this problem, a magneto-resistance effect sensor has been focused that is used for HDDs (Hard Disk Drives) or the like, it has been reported that measurement of a minute magnetic field is possible of equal to or less than 100 pT (pica Tesla) that is required in magnetocardiogram measurement. In a MR (Magneto-Resistive) sensor, it is a problem that a large 1/f noise easily occurs when a low frequency magnetic field is measured of 1 to 1000 Hz that is essential to a biomagnetic application. In a HDD magnetic head, it has not been a problem since a high frequency signal of one MHz or more is dealt with.

It has been known that an increase of volume of sensing magnetic material and removal of magnetic non-uniformity (linear response with no hysteresis He) are effective in reduction of the 1/f noise. It has been reported that magnetic field detection sensitivity same as the sensitivity of a current-perpendicular-to-plane tunnel magneto-resistance effect sensor (TMR sensor) of a large MR ratio is obtained by using a current-in-plan anisotropic magneto-resistance effect sensor (also referred to as an AMR sensor) in which the increase of volume of magnetic material is easy even when the MR ratio is small. In the TMR sensor, the 1/f noise is also a problem that is unique to a tunnel barrier.

In addition, a GMR (Giant Magneto-Resistive) sensor has been known that uses a free layer in which crystalline alloys of CoFe and NiFe are layered. However, when the reduction of the 1/f noise is aimed by thickening a NiFe layer of the free layer, a decrease in the MR ratio is more significant than a decrease in the 1/f noise, and an S/N ratio is not improved, and the magnetic field detection sensitivity is not improved.

### BRIEF DESCRIPTION OF THE DRAWINGS

FIG. 1 is a plan view showing a magnetic sensor of a first embodiment.

FIG. 2 is a sectional view cut by a cutting plane line A-A shown in FIG. 1.

FIG. 3 is a sectional view showing a magneto-resistive film of the magnetic sensor of the first embodiment.

FIG. 4 is a diagram showing a relationship between a thickness of a second magnetic field detection layer and noise and output in the magnetic sensor of the first embodiment.

FIG. 5 is a diagram showing a relationship between a thickness of a second magnetic field detection layer and noise and output in a magnetic sensor of a comparative example.

FIG. 6 is a diagram showing a relationship between a SN ratio and a thickness in a case in which an amorphous magnetic alloy is used as a second magnetic field detection layer in a magnetic sensor for a magnetic head.

FIG. 7 is a diagram for explaining a relationship between noise and output and a current path length of the magnetic sensor of the first embodiment.

FIG. 8 is a diagram showing a magnetic sensor apparatus of a second embodiment.

FIG. 9 is a diagram showing a magnetic sensor apparatus of a third embodiment.

FIG. 10 is a diagram showing a magnetic sensor apparatus of a first modification of the third embodiment.

FIG. 11 is a diagram showing a magnetic sensor apparatus of a second modification of the third embodiment.

### DETAILED DESCRIPTION

A magnetic sensor according to an embodiment includes: a magneto-resistive film including a laminate structure, the laminate structure including a first magnetic layer, a second magnetic layer, and an intermediate layer arranged between the first magnetic layer and the second magnetic layer; and a pair of electrodes for supplying current in a first direction perpendicular to a laminate direction of the magneto-resistive film, wherein the second magnetic layer includes an amorphous magnetic layer, and a crystalline magnetic layer arranged between the amorphous magnetic layer and the intermediate layer, and a length of a current path of the magneto-resistive film is 10 μm or more.

Embodiments are described below with reference to the drawings.

### First Embodiment

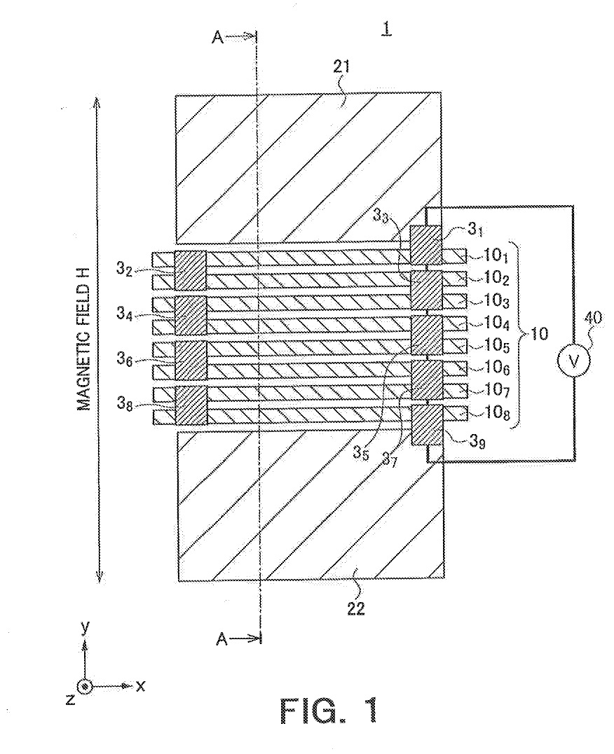

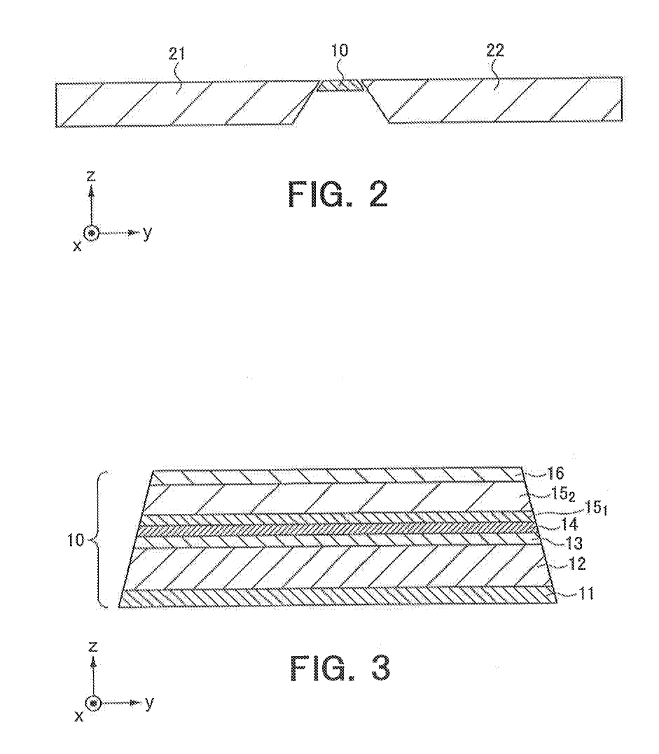

A magnetic sensor of a first embodiment is described with reference to FIG. 1 to FIG. 3. FIG. 1 shows a plan view of a magnetic sensor 1 of the first embodiment, and FIG. 2 shows a sectional view cut by a cutting plane line A-A shown in FIG. 1, and FIG. 3 shows a sectional view of a magneto-resistive film 10 of the magnetic sensor 1.

The magnetic sensor 1 of the first embodiment includes the magneto-resistive film 10, and magnetic field concentrators 21, 22. The magneto-resistive film 10 has a laminate structure on which a base layer 11, an antiferromagnetic layer 12, a magnetization pinned layer 13, an intermediate layer 14, a first magnetic field detection layer 15₁, a second magnetic field detection layer 15₂, and a cap layer 16 are sequentially formed on a substrate not shown. The base layer 11 is formed from, for example, Ta, Ru, or Cu. The antiferromagnetic layer 12 is formed from, for example, IrMn, and pins magnetization of the magnetization pinned layer 13. The magnetization pinned layer 13 is formed from, for example, CoFe. The intermediate layer 14 is formed from a nonmagnetic metal, for example, Cu. Materials of the first and second magnetic field detection layers 15₁, 15₂ are described later. The cap layer 16 is formed from, for example, Ru, Ta, or Cu. The magneto-resistive film 10 is also referred to as a GMR film since its intermediate layer is formed form a nonmagnetic metal.

The magneto-resistive film 10 is patterned into a desired shape. For example, to realize an appropriate resistance suitable for sensor operation, for example, from 100Ω to 10 kΩ, the magneto-resistive film 10 is patterned into a rectangular shape in which a current direction is a longitudinal direction (x. direction). For example, the magneto-resistive film 10 is patterned into a rectangular shape of a length of from 0.01 mm to 5 mm, and a width of from 1 μm to 100 μm. In FIG. 1, the magneto-resistive film 10 has a configuration in which the magneto-resistive film 10 is divided into plural (eight) rectangular shapes. That is, the magneto-resistive film 10 shown in FIG. 1 is divided into eight magneto-resistive parts 10₁ to 10₈. Incidentally, the magneto-resistive film 10 may be one magneto-resistive part. The magneto-resistive film 10 shown in FIG. 2 represents one magneto-resistive part.

Nine electrodes 3₁ to 3₉ are provided so that these eight magneto-resistive parts 10₁ to 10₈ are connected to each other in series. The electrode 3₁ is provided in a vicinity of a right end of the magneto-resistive part 10₁, and the electrode 3₂ᵢ(i=1, 2, 3, 4) connects a vicinity of a left end of the magneto-resistive part 10₂ᵢ₋₁ and a vicinity of a left end of the magneto-resistive part 10₂ᵢ to each other. The electrode 3₂ᵢ₊₁ (i=1, 2, 3) connects a vicinity of a right end of the magneto-resistive part 10₂ᵢ and a vicinity of a right end of the magneto-resistive part 10₂ᵢ₋₁ to each other. The electrode 3₉ is connected to a vicinity of a right end of the magneto-resistive part 10₈. That is, each magneto-resistive part 10₁ (i=1, . . . , 9) is connected to a pair of electrodes. In addition, the electrode 3₁ and the electrode 3₉ are connected to a circuit 40 that applies a voltage for supplying current to the magneto-resistive film 10. With the circuit 40, the current flows between the pair of electrodes of each magneto-resistive part 10ᵢ (i=1, . . . , 9), and an area between the pair of electrodes is a magnetic field detection area.

To reduce influence of noise due to a magnetic domain caused in an edge portion in a longitudinal direction of each of the first and second magnetic field detection layers 15₁, 15₂, each of the electrodes 3₁ to 3₉ may be provided to a position apart to some extent from an edge of the magneto-resistive part, not to a strict edge portion of each magneto-resistive part 10ᵢ (i=1, . . . , 8).

Since magnetic fields to be measured has a uniform magnetic field area of about a few mm, a pair of magnetic concentrators 21, 22 made of a high permeability soft magnetic material, which collects signal magnetic flux into the first and second magnetic field detection layers 15₁, 15₂, is provided at each end in a width direction (y direction) of the magneto-resistive film 10. The magnetic field concentrators 21, 22 are also referred to as magnetic flux concentrators (MFCs) 21, 22, For each of the MFCs 21, 22, for example, NiFe, NiFeMoCu, or a Co-based amorphous alloy is used. It is preferable that a thickness (length in the z direction) of each of the MFCs 21, 22 is made to be sufficiently thicker than a thickness of each of the first and second magnetic field detection layers 15₁, 15₂ (for example, a few micrometers thickness or more), and further, each of the MFCs 21, 22 has a tapered shape in which the thickness of each of the MFCs 21, 22 is gradually thinner in a vicinity of a junction of the first and second magnetic field detection layers 15₁, 15₂. With such a tapered shape, improvement of concentration efficiency of the signal magnetic flux and a sensitivity increase can be obtained.

For the first magnetic field detection layer 15₁, an alloy is used that contains at least two elements from a group of Co, Fe, and Ni, which are suitable for expression of GMR, for example, a crystalline magnetic alloy such as CoFe, NiFe, or CoFeNi.

For the second magnetic field detection layer 15₂, an amorphous magnetic alloy is used, for example, an amorphous alloy such as CoFeSiB, or CoXY. Here, X represents Zr or Hf, and Y represents Ta or Nb. Since the amorphous magnetic alloy does not have long-period atomic arrangement periodicity, a crystalline magnetic anisotropy is substantially zero. In addition, by appropriately adjusting composition of the magnetic alloy, magnetostriction can be made to be roughly zero, and an excellent soft magnetic property can be obtained, and magnetic noise can be suppressed. Further, the amorphous magnetic alloy, in comparison with a resistivity p (from 10 μΩcm to 30 μΩcm) of the first magnetic field detection layer 15₁, has a large resistivity of roughly equal to or less than 100 μΩcm, so that the current is concentrated in an expression portion of magneto-resistance, and a decrease in the MR ratio can be reduced.

### First Example

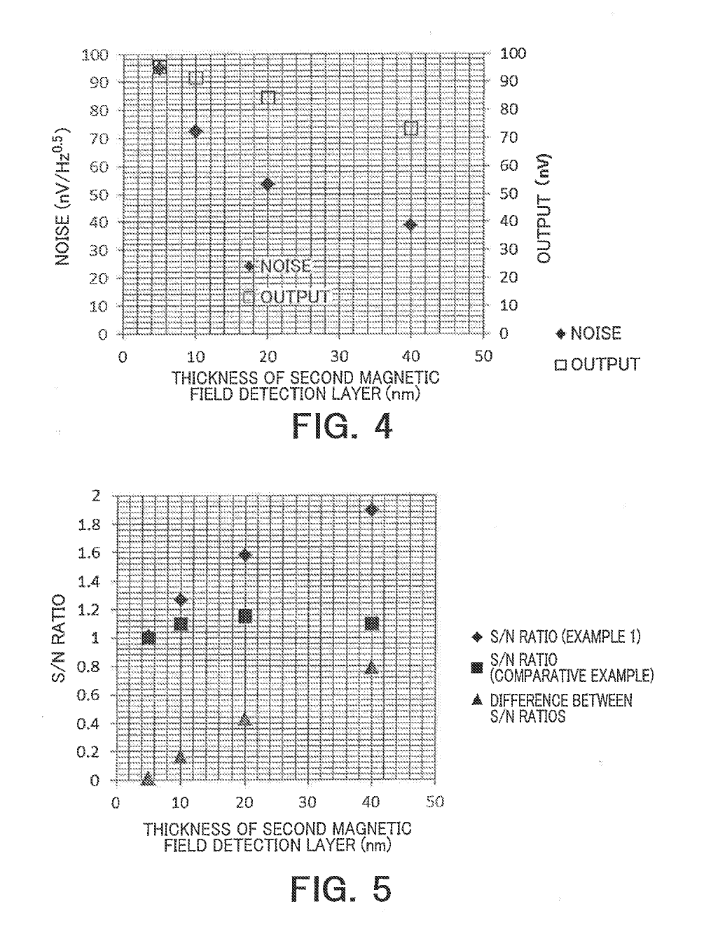

Next, a result of examination by an experiment is shown in FIG. 4 of a relationship between a thickness of the second magnetic field detection layer 15₂ and noise and output in the magnetic sensor 1 of the first embodiment. The magnetic sensor 1 used for the experiment is configured by the magneto-resistive film 10 having eight magneto-resistive part, and each magneto-resistive part has a length of 1.2 mm, and a width of 60 μm. As the MFCs 21, 22, NiFeMoCu of a thickness of 10 μm has been used. An amplification factor of a signal magnetic field by each of the MFCs 21, 22 is 500 times. An input voltage is 5V, and the output is a detection value of a magnetic field of 1 pT. In addition, the noise is a voltage at a signal magnetic field of zero measured by a spectrum analyzer, and is a value at a frequency of 10 Hz. Further, a heat treatment condition or the like has been adjusted so that a saturation magnetic field of each of the first and second magnetic field detection layers 15₁, 15₂ is 25 Oe. As the first magnetic field detection layer 15₁, CoFe of a thickness of 2 mm has been used. As the second magnetic field detection layer 15₂, a CoFeSiB amorphous alloy has been used.

As can be seen from FIG. 4, when the thickness of the second magnetic field detection layer 15₂ is increased, the output and noise are decreased, and in particular the noise is significantly decreased. FIG. 5 shows a relationship between a SN ratio, that is, a ratio of the voltages of the output and noise shown in FIG. 4, and the thickness of the second magnetic field detection layer 15₂.

As a comparative example, a magnetic sensor has been created that has the same configuration as that of the first example except for using a NiFe alloy as the second magnetic field detection layer 15₂. A result of examination by an experiment is also shown in FIG. 5 of a relationship between a SN ratio of the magnetic sensor of the comparative example and the thickness of the second magnetic field detection layer 15₂. In addition, FIG. 5 also shows a difference of the SN ratio of the magnetic sensor between the first example and the comparative example. As can be seen from FIG. 5, in a case of the magnetic sensor of the comparative example in which the NiFe alloy is used as the second magnetic field detection layer 15₂, there is no apparent change in the SN ratio when the thickness of the second magnetic field detection layer 15₂ is increased.

On the other hand, in a case of using the amorphous magnetic alloy as the second magnetic field detection layer 15₂ as in the first embodiment, the SN ratio is increased when the thickness of the second magnetic field detection layer 15₂ is increased. That is, when the amorphous magnetic alloy is used as the second magnetic field detection layer 15₂ as in the present embodiment, the SN ratio has a margin, so that high sensitivity detection of minute magnetic field is possible. An increase effect of the SN ratio in comparison with the comparative example is apparent when the thickness of the second magnetic field detection layer 15₂ is 10 nm or more.

Next, a magnetic sensor of the first embodiment has been produced whose size is changed for a HDD magnetic head, to be mounted on the magnetic head. For example, since the magnetic sensor for the magnetic head reads a micro-bit medium magnetic field, a length (recording track width) of the magneto-resistive part configuring the magneto-resistive film is approximately 0.1 μm, which is significantly smaller than that of a magnetic sensor for a living body.

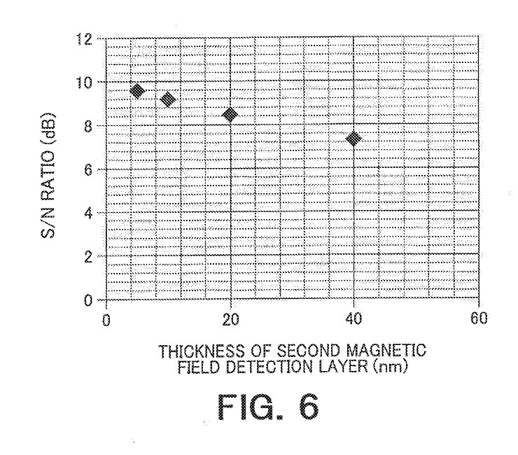

FIG. 6 shows a result obtained by an experiment of a relationship between the thickness and the SN ratio in a case in which the amorphous magnetic alloy is used as the second magnetic field detection layer in the magnetic sensor for the magnetic head. As can be seen from FIG. 6, in the magnetic sensor for the magnetic head, different from the first embodiment, when the thickness of the second magnetic field detection layer is increased, the SN ratio is decreased, that is, read performance is degraded.

The magnetic sensor for the magnetic head detects a high frequency magnetic field of one MHz or higher, and 1/f noise is a sufficiently small value since the 1/f noise is inversely proportional to the frequency. In the magnetic sensor for the magnetic head, since another noise is primary, the SN ratio is decreased due to an output decrease. An effect of using the amorphous magnetic alloy as the second magnetic field detection layer is apparent in the magnetic sensor for the living body or the like that detects a low frequency of around 10 Hz or less.

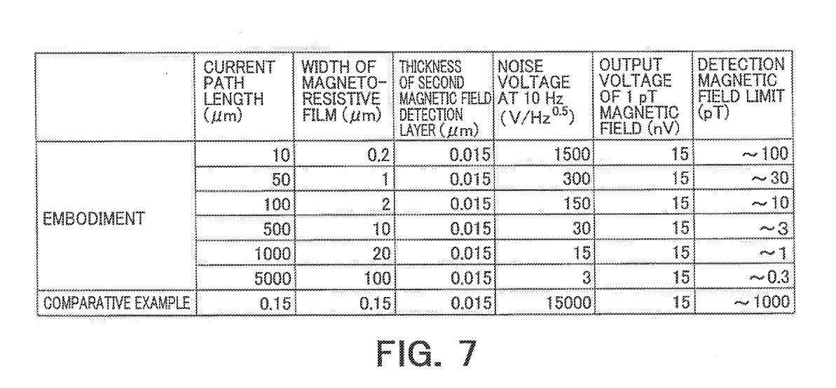

Next, a result is shown in FIG. 7 in which: plural samples have been produced that have different current path lengths in the longitudinal direction in which current flows through the magneto-resistive film, and have different widths of the magneto-resistive film, in the magnetic sensor of the first embodiment; and output at 1 pT, noise at 10 Hz, and a detection limit magnetic field at which the output and the noise coincide with each other have been obtained. In these samples, the width of the magneto-resistive film has been increased with the increase of the current path length so that sensor resistance is approximately 1000Ω, which is preferable from a viewpoint of consumption current, and Johnson noise suppression.

As can be seen from FIG. 7, when the current path length is increased, the output is not changed, but the noise is reduced. This is because the 1/f noise is inversely proportional to a root value of a total volume of the first and second magnetic field detection layers. As a result, a detection magnetic field limit is decreased with a volume increase of each of the first and second magnetic field detection layers, and a high sensitivity magnetic sensor can be achieved.

A result is shown in FIG. 7 in which; a magnetic sensor has been produced that is used for the HDD as a comparative example; and similarly, output at 1 pT, noise at 10 Hz, and a detection limit magnetic field D at which the output and the noise coincide with each other have been obtained. Incidentally, in the magnetic sensor of the comparative example, the current path length is 0.15 μm.

As can be seen from FIG. 7, in the comparative example, a magnetic field of 1000 pT or less cannot be detected, and detection of approximately 100 pT is impossible that is necessary for biomagnetism detection such as magnetocardiograph or magnetoencephalograph. That is, it can be seen that, for magnetic field detection of 100 pT or less, a magneto-resistive film is required that has a current path length of 10 μm or more.

As described above, with the first embodiment, a magnetic sensor can be provided in which a decrease in an MR ratio is small, and that is capable of reducing the 1/f noise.

### Second Embodiment

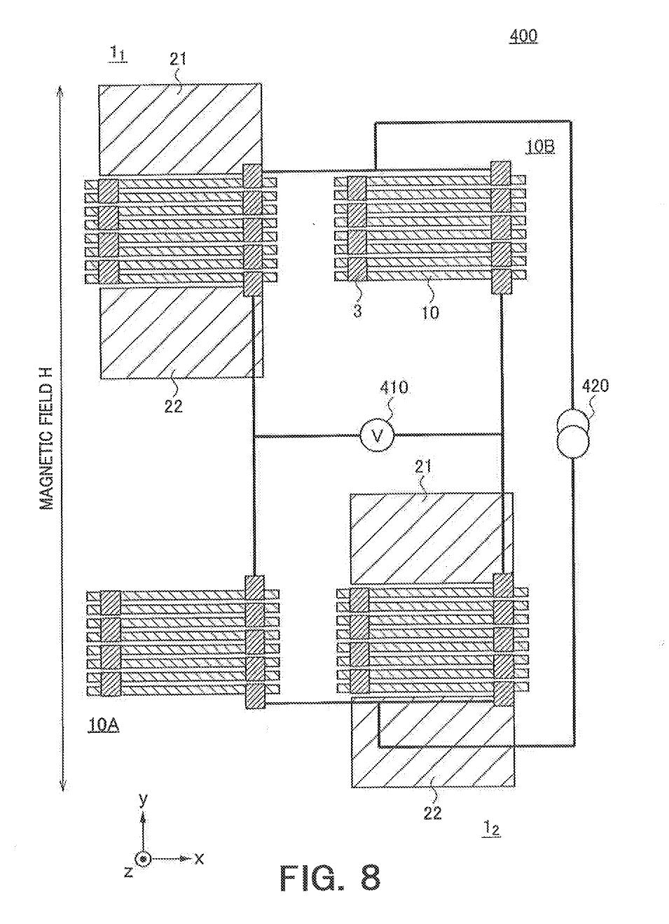

Next, a magnetic sensor apparatus of a second embodiment is shown in FIG. 8. A magnetic sensor apparatus 400 of the second embodiment includes: two magnetic sensors 1₁, 1₂ each having the same configuration as that of the magnetic sensor 1 shown in FIG. 1; two magneto-resistive films 10A, 10B each having the same configuration as that of the magneto-resistive film 10 of the magnetic sensor 1 shown in FIG. 1 except for MFC; a voltmeter 410; and a current source 420. The magnetic sensor 1₁ and the magneto-resistive film 10A are connected to each other in series to configure a first current line. The magnetic sensor 1₂ and the magneto-resistive film 10B are connected to each other in series to configure a second current line. The first current line and the second current line are connected in parallel to the current source 420. Thus, current flows through the magnetic sensor 1₁ and the magneto-resistive film 10A of the first current line and the magnetic sensor 1₂ and the magneto-resistive film 10B of the second current line. A signal magnetic field is detected by the magnetic sensors 1₁, 1₂.

When MFCs 21, 22 are selected so that a gain of signal magnetic field concentration is 100 times or more in the magnetic sensors 1₁, 1₂, each of the magneto-resistive films 10A, 10B can be regarded as a fixed resistance whose resistance does not change. The magnetic sensor 1₁ of the first current line and the magnetic sensor 1₂ of the second current line are separately arranged in a current upstream and downstream. With such a configuration, a resistance of each of the magnetic sensors 1₁, 1₂ is changed in accordance with the signal magnetic field, and a potential difference is generated between intermediate portions of the first and second current lines, and an output voltage is obtained. The output voltage is detected by the voltmeter 410.

Incidentally, similarly to a configuration of a normal bridge circuit, each of the magneto-resistive films 10A, 10B may be a fixed resistance made of a nonmagnetic material whose resistance is not changed due to the magnetic field.

As described above, with the second embodiment, the magnetic sensor of the first embodiment is used, so that a magnetic sensor apparatus can be provided in which a decrease in an MR ratio is small, and that is capable of reducing 1/f noise.

### Third Embodiment

Next, the magnetic sensor of the first embodiment can be used for a magnetoencephalograph that detects a magnetic field generated by a cranial nerve. This is described as a third embodiment.

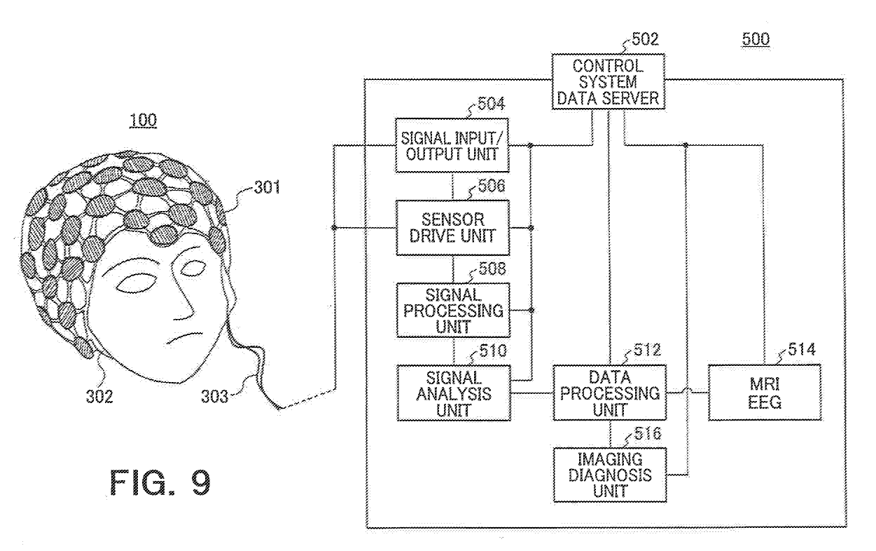

A magnetic sensor apparatus of the third embodiment is described with reference to FIG. 9. A magnetic sensor apparatus 100 of the third embodiment is a magnetoencephalograph, and a left side figure in FIG. 9 schematically shows a state in which the magnetoencephalograph 100 is worn on a head of a human body. The magnetoencephalograph 100 has a configuration in which plural sensor units, for example, about 100 sensor units 301, are installed on a flexible base body 302.

In each of the sensor, units 301, one magnetic sensor may be arranged of the magnetic sensor of the first embodiment, and the plural magnetic sensors may be arranged. The plural magnetic sensors may configure a circuit such as of differential detection, and another sensor such as a potential terminal or an acceleration sensor may be installed simultaneously. The magnetic sensor of the first embodiment can be made to be very small in comparison with a conventional SQUID magnetic sensor, so that installation of the plural sensor units, installation of the circuit, and coexistence with another sensor are easy.

The flexible base body 302 is made of, for example, an elastic body such as a silicone resin, and is configured to connect the sensor units 301 to each other in a belt shape and to be capable of being snugly fitted with the head. The base body 302 may be a base body obtained by processing contiguous film in a hat shape; however, a net-shaped base body shown in FIG. 9 is preferable, which has an excellent wearability and improves snug fit with a human body.

An input/output cord 303 of the sensor units 301 is connected to a sensor drive unit 506 and a signal input/output unit 504 of a diagnosis apparatus 500. The sensor units 301 performs predetermined magnetic field measurement based on power from the sensor drive unit 506 and a control signal from the signal input/output unit 504, and a result of the measurement is input to the signal input/output unit 504 in parallel. The signal obtained by the signal input/output unit 504 is then transmitted to a signal processing unit 508, and is subjected to processing such as noise removal, filtering, amplification, and signal operation, in the signal processing unit 508. After that, the signal is subjected to signal analysis in which a particular signal is extracted for magnetoencephalogram measurement, and signal phases are matched to each other, in a signal analysis unit 510. Data in which the signal analysis has been completed is transmitted to a data processing unit 512. In the data processing unit 512, image data such as magnetic resonance imaging (MRI) and scalp potential information such as electroencephalogram (EEG) are incorporated, and data analysis is performed, such as neural ignition point analysis and inverse problem analysis. A result of the analysis is transmitted to an imaging diagnosis unit 516, and imaging is performed to facilitate diagnosis. The above series of operation is controlled by a control system 502, and necessary data such as primary signal data or metadata during data processing is stored in a data server. Incidentally, as shown in FIG. 9, the data server and the control system may be integrated.

In the third embodiment shown in FIG. 9, the sensor units 301 are installed on the human body head; however, when the units are installed on a human body chest, magnetocardiogram measurement is possible. In addition, when the units are installed on an abdomen of a pregnant woman, it can be used for a heart rate test of a fetus.

An entire of the magnetic sensor apparatus including a subject is preferably installed in a shield room to prevent the geomagnetism and magnetic noise. Alternatively, a system may be provided for locally shielding a measurement site of the human body and the sensor units 301. In addition, a shield system may be provided to the sensor units 301, and an effective shielding may be performed in the signal analysis and the data processing.

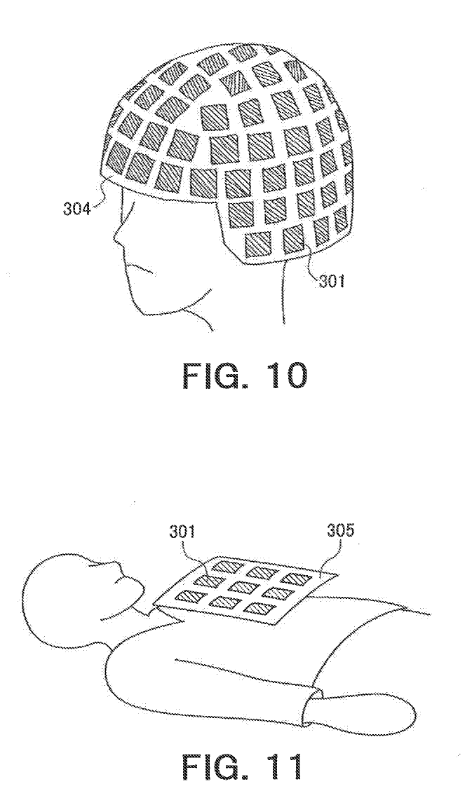

In the magnetic sensor 100 shown in FIG. 9, the sensor units 301 each including a high sensitivity magnetic sensor are installed to the flexible base body 302; however, the units can be installed to a fixed base body such as in a conventional magnetoencephalograph or magnetocardiograph. Examples are shown in FIG. 10 and FIG. 11. FIG. 10 shows an example of the magnetoencephalograph, and the sensor units 301 are installed on a helmet-shaped hard base body 304. FIG. 11 is an example of the magnetocardiograph, and the sensor units 301 are installed on a plate-shaped hard base body 305. In both cases, input/output of a signal from the sensor units 301 and processing of the signal are the same as those in FIG. 9.

In addition, as the sensor unit 301, a magnetic sensor apparatus 400 shown in FIG. 8 may be used.

As described above, with the third embodiment, the magnetic sensor of the first embodiment or the magnetic sensor apparatus of the second embodiment is used as the sensor unit, so that a magnetic sensor apparatus can be provided in which a decrease in an MR ratio is small, and that is capable of reducing 1/f noise.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

1. A magnetic sensor comprising:
a magneto-resistive film including a laminate structure, the laminate structure including a first magnetic layer, a second magnetic layer, and an intermediate layer arranged between the first magnetic layer and the second magnetic layer; and
a pair of electrodes for supplying current in a first direction perpendicular to a laminate direction of the magneto-resistive film, wherein
the second magnetic layer includes an amorphous magnetic layer, and a crystalline magnetic layer arranged between the amorphous magnetic layer and the intermediate layer, and a length of a current path of the magneto-resistive film is 10 μm or more.

2. The sensor according to claim 1, wherein
the magneto-resistive film includes a plurality of magneto-resistive parts connected to each other in series, and each of the magneto-resistive parts includes the first magnetic layer, the intermediate layer, the crystalline magnetic layer, and the amorphous magnetic layer.

3. The sensor according to claim 1, wherein
the amorphous magnetic alloy layer has a thickness in the laminate direction of 10 nm or more.

4. The sensor according to claim 1, further comprising
a pair of magnetic films arranged at a side portion of the magneto-resistive film, wherein each of the magnetic films has a thickness in the laminate direction thicker than a thickness of each of the amorphous magnetic layer and the crystalline magnetic layer.

5. The sensor according to claim 1, wherein
the amorphous magnetic layer contains CoFeSiB or CoXY, wherein X represents at least one of Zr and Hf, and Y represents at least one of Ta and Nb.

6. The sensor according to claim 1, wherein
the crystalline magnetic layer is an alloy containing at least two elements of Co, Fe, and Ni.

7. A magnetic sensor apparatus comprising:
first and second magnetic sensors according to claim 1; first and second resistors; and a voltmeter, wherein the first magnetic sensor and the first resistor are connected to each other in series to configure a first serial portion, the second magnetic sensor and the second resistor are connected to each other in series to configure a second serial portion, the first serial portion and the second serial portion are connected to each other in parallel, and the voltmeter measures a potential difference between a connection node between the first magnetic sensor and the first resistor and a connection node between the second magnetic sensor and the second resistor.

8. The apparatus according to claim 7, wherein
the magneto-resistive film includes a plurality of magneto-resistive parts connected to each other in series, and each of the magneto-resistive parts includes the first magnetic layer, the intermediate layer, the crystalline magnetic layer, and the amorphous magnetic layer.

9. The apparatus according to claim 7, wherein
the amorphous magnetic alloy layer has a thickness in the laminate direction of 10 nm or more.

10. The apparatus according to claim 7, further comprising
a pair of magnetic films arranged at a side portion of the magneto-resistive film, wherein each of the magnetic films has a thickness in the laminate direction thicker than a thickness of each of the amorphous magnetic layer and the crystalline magnetic layer.

11. The apparatus according to claim 7, wherein
the amorphous magnetic layer contains CoFeSiB or CoXY, wherein X represents at least one of Zr and Hf, and Y represents at least one of Ta and Nb.

12. The apparatus according to claim 7, wherein
the crystalline magnetic layer is an alloy containing at least two elements of Co, Fe, and Ni.

13. A magnetic sensor apparatus comprising:
a magnetic sensor according to claim 1; and
a diagnosis apparatus including a processing analysis circuit for processing and analyzing a magnetic field detection signal from the magnetic sensor, and an imaging circuit for imaging an analysis result of the processing analysis circuit.

14. The apparatus according to claim 13, wherein
the magnetic sensor detects a magnetic field from a brain.

15. The apparatus according to claim 13, wherein
the magnetic sensor detects a magnetic field from a heart.

16. The apparatus according to claim 13, wherein
the magneto-resistive film includes a plurality of magneto-resistive parts connected to each other in series, and each of the magneto-resistive parts includes the first magnetic layer, the intermediate layer, the crystalline magnetic layer, and the amorphous magnetic layer.

17. The apparatus according to claim 13, wherein
the amorphous magnetic alloy layer has a thickness in the laminate direction of 10 nm or more.

18. The apparatus according to claim 13, further comprising
a pair of magnetic films arranged at a side portion of the magneto-resistive film, wherein each of the magnetic films has a thickness in the laminate direction thicker than a thickness of each of the amorphous magnetic layer and the crystalline magnetic layer.

19. The apparatus according to claim 13, wherein
the amorphous magnetic layer contains CoFeSiB or CoXY, wherein X represents at least one of Zr and Hf, and Y represents at least one of Ta and Nb.

20. The apparatus according to claim 13, wherein
the crystalline magnetic layer is an alloy containing at least two elements of Co, Fe, and Ni.
